(19) United States
(12) Patent Application Publication
(10) Pub. No.: US 2023/0255532 A1
(43) Pub. Date: Aug. 17, 2023

(54) SYSTEM AND METHOD FOR FUNCTIONAL ASSESSMENT OF URINARY TRACT USING MAGNETIC RESONANCE IMAGING

(71) Applicant: WISCONSIN ALUMNI RESEARCH FOUNDATION, Madison, WI (US)

(72) Inventors: Alejandro Roldán-Alzate, Middleton, WI (US); Wade Bushman, Middletown, RI (US)

(21) Appl. No.: 17/669,419

(22) Filed: Feb. 11, 2022

(51) Int. Cl. A61B 5/20; G01R 33/385; G01R 33/56; A61B 5/055; G06T 7/12; G06T 7/00

(52) U.S. Cl. CPC A61B 5/202; G01R 33/385; G01R 33/5608; A61B 5/055; A61B 5/205; A61B 5/204; G06T 7/12; G06T 7/0012; G06T 2207/10088; G06T 2207/30004

(57) ABSTRACT

A system and method includes receiving time-resolved images of a urinary tract of a subject as a bladder of the urinary tract begins, continues through, and completes a dynamic process involving a bladder and segmenting the time-resolved images of the urinary tract to identify boundaries of anatomical structures of the urinary tract. The method further includes performing a surface mapping of the boundaries of the anatomical structures to produce a consistent set of mapped anatomical structures across the time-resolved images, using a flow model and the consistent set of mapped anatomical structures, calculating metrics describing function of the urinary tract during the dynamic process, and generating a report using the metrics describing function of the urinary tract during the dynamic process.

### BACKGROUND

The present disclosure relates to systems and methods for assessing biological functions and, more particularly, to systems and methods for evaluating and quantifying urinary tract function non-invasively using magnetic resonance imaging (MM).

As people age, the urinary tract can exhibit many changes in function that produce a variety of lower urinary tract symptoms (LUTS). The result is that a majority of men and women over the age of 60 experience significantly bothersome LUTS. They present with a wide range of urological symptoms that include storage symptoms of urinary frequency, urgency, voiding symptoms of diminished stream and incomplete bladder emptying and various forms of urinary incontinence. Standard clinical evaluation of these patients includes a history and physical examination. Usually, this is the extent of the evaluation and treatment is prescribed based on an empiric diagnosis. Specialists in Urology and Gynecology will sometimes perform a measure of urinary flow rate and a determination of post-void bladder residual volume.

A more comprehensive assessment of bladder function sometimes utilized by specialists is multi-channel urodynamic studies. See V. Nitti, Pressure Flow Urodynamic Studies: The Gold Standard for Diagnosing Bladder Outlet Obstruction, Rev Urol. 2005; 7(Suppl 6): S14-S21. This is a procedure using catheters placed within the bladder and rectum to measure bladder pressure and flow during voiding and allow the clinician use calculated indices to diagnose conditions such as outlet obstruction or impaired bladder contractility. Although being considered “state of the art”, multi-channel urodynamic studies have several significant limitations and drawbacks. The assessment of bladder function is indirect, based on measurements of pressure and flow during voiding and the study provides no anatomic information. In hospital settings with extensive resources, urodynamic studies can be performed in combination with fluoroscopic imaging but even then, the anatomic detail provided is extremely limited. The performance of multi-channel urodynamics requires specially trained staff and a considerable investment in dedicated equipment. For this reason, multi-channel urodynamics is something that most urologists and gynecologists in private practice can afford and offer to their patients. Finally, and perhaps most importantly, multi-channel urodynamic studies are invasive and uncomfortable for the patients. A non-invasive study that provides pressure and flow measurements with more anatomic information and can be made available to all urologists and gynecologists is clearly needed.

### SUMMARY OF THE DISCLOSURE

The present disclosure provides systems and methods for assessing urodynamics non-invasively. In one configuration, a system and method is provided that uses MRI data acquired throughout bladder function as an input to a computational fluid dynamics (CFD) model of a dynamic process. The CFD model uses the MM data to generate functional continuous metrics non-invasively. The spectrum of metrics can be used to generate one or more indexes, for example, such as those currently user to drive clinical care protocols. By generating functional MR images, and a spectrum of functional metrics, the clinician can be provided with information that matches the current gold-standard of clinical care and then far exceeds that standard.

In accordance with one aspect of the disclosure, a magnetic resonance (MR) imaging system is provided that includes a magnet system configured to generate a static magnetic field (BO) about at least a portion of a subject including a urinary tract, a plurality of gradient coils configured to apply magnetic gradients to the static magnetic field, and a radio frequency (RF) system configured to apply an excitation field to the subject and acquire time-resolved, three-dimensional (3D) MR image data from the urinary tract of the subject. The system also includes a computer system programmed to control the plurality of gradient coils and the RF system to acquire the time-resolved 3D MR image data from the urinary tract as a bladder of the urinary tract begins, continues through, and completes a dynamic process involving a bladder. The computer system is further programmed to reconstruct the time-resolved 3D MR image data to produce volumetric, time-resolved images of the urinary tract, segment the volumetric, time-resolved images of the urinary tract to identify anatomical structures of the urinary tract, and perform a surface mapping of the anatomical structures to produce a consistent set of mapped anatomical structures across the volumetric, time-resolved images. The computer system is also programmed to process the consistent set of mapped anatomical structures using a flow model to calculate metrics describing a function of the urinary tract during the dynamic process, generate a report using the metrics describing function of the urinary tract during the dynamic process, and display the report for clinical analysis of the function of the urinary tract.

In accordance with another aspect of the disclosure, a method is provided that includes receiving time-resolved images of a urinary tract of a subject as a bladder of the urinary tract begins, continues through, and completes a dynamic process involving a bladder and segmenting the time-resolved images of the urinary tract to identify boundaries of anatomical structures of the urinary tract. The method further includes performing a surface mapping of the boundaries of the anatomical structures to produce a consistent set of mapped anatomical structures across the time-resolved images, using a flow model and the consistent set of mapped anatomical structures, calculating metrics describing function of the urinary tract during the dynamic process, and generating a report using the metrics describing function of the urinary tract during the dynamic process.

These and other advantages and features of the invention will become more apparent from the following detailed description of the preferred embodiments of the invention when viewed in conjunction with the accompanying drawings.

### DESCRIPTION OF THE DRAWINGS

FIG. 1 is a block diagram of an exemplary magnetic resonance imaging (MM) system configured in accordance with the present disclosure.

FIG. 2 is a flow chart setting forth some non-limiting examples of steps of a process in accordance with the present disclosure.

FIG. 3A is a set of adjacent images being mapped in accordance with the present disclosure.

FIG. 3B shows a subsequent one of the adjacent images of FIG. 3A being partitioned and processed.

FIG. 3C shows a mapped surface produced carrying out the process described with respect to FIGS. 3B and 3C.

FIG. 4A is a non-limiting example visualizations, including images or movies, that can be included in a report generated in accordance with the present disclosure.

FIG. 4B is a set of metrics determined in accordance with the present disclosure that extend over time and can be divided by anatomy.

### DETAILED DESCRIPTION

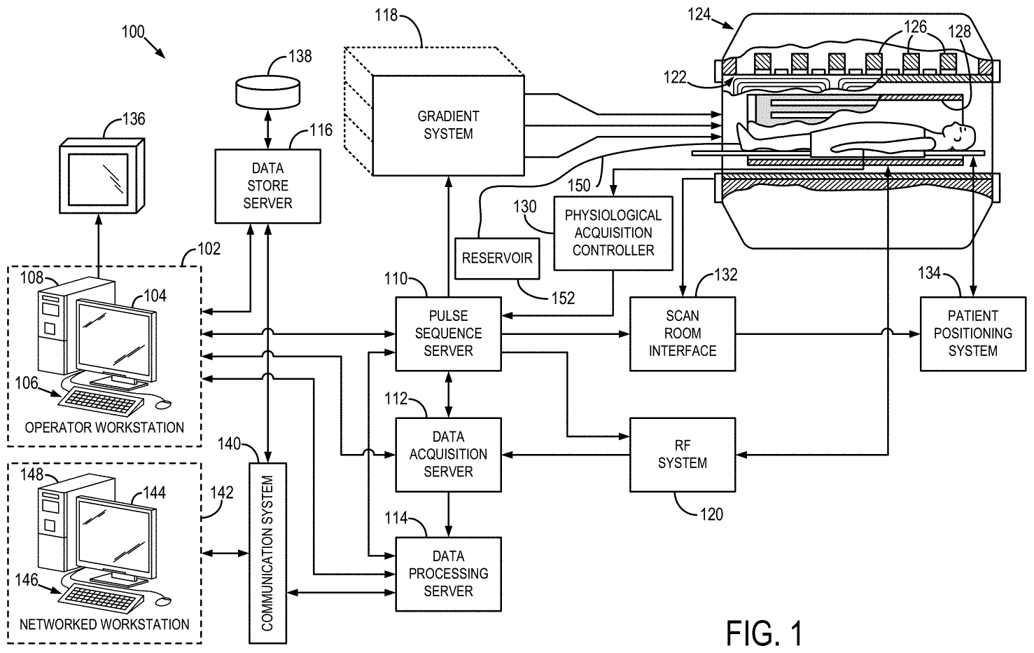

Referring now to FIG. 1, a magnetic resonance imaging (Mill) system 100 is provided that may be configured, programmed, or otherwise utilized in accordance with the present disclosure. The MM system 100 includes an operator workstation 102, which will typically include a display 104, one or more input devices 106 (such as a keyboard and mouse or the like), and a processor 108. The processor 108 may include a commercially available programmable machine running a commercially available operating system. The operator workstation 102 provides the operator interface that enables scan prescriptions to be entered into the MM system 100. In general, the operator workstation 102 may be coupled to multiple servers, including a pulse sequence server 110; a data acquisition server 112; a data processing server 114; and a data store server 116. The operator workstation 102 and each server 110, 112, 114, and 116 are connected to communicate with each other. For example, the servers 110, 112, 114, and 116 may be connected via a communication system 140, which may include any suitable network connection, whether wired, wireless, or a combination of both. As an example, the communication system 140 may include both proprietary or dedicated networks, as well as open networks, such as the internet.

The pulse sequence server 110 functions in response to instructions downloaded from the operator workstation 102 to operate a gradient system 118 and a radiofrequency (RF) system 120. Gradient waveforms to perform the prescribed scan are produced and applied to the gradient system 118, which excites gradient coils in an assembly 122 to produce the magnetic field gradients Gₓ, G_{y}, G_{z} used for position encoding magnetic resonance signals. The gradient coil assembly 122 forms part of a magnet assembly 124 that includes a polarizing magnet 126 and a whole-body RF coil 128.

RF waveforms are applied by the RF system 120 to the RF coil 128, or a separate local coil (not shown in FIG. 1), in order to perform the prescribed magnetic resonance pulse sequence. Responsive magnetic resonance signals detected by the RF coil 128, or a separate local coil, are received by the RF system 120, where they are amplified, demodulated, filtered, and digitized under direction of commands produced by the pulse sequence server 110. The RF system 120 includes an RF transmitter for producing a wide variety of RF pulses used in Mill pulse sequences. The RF transmitter is responsive to the scan prescription and direction from the pulse sequence server 110 to produce RF pulses of the desired frequency, phase, and pulse amplitude waveform. The generated RF pulses may be applied to the whole-body RF coil 128 or to one or more local coils or coil arrays.

The RF system 120 also includes one or more RF receiver channels. Each RF receiver channel includes an RF preamplifier that amplifies the magnetic resonance signal received by the coil 128 to which it is connected, and a detector that detects and digitizes the I and Q quadrature components of the received magnetic resonance signal. The magnitude of the received magnetic resonance signal may, therefore, be determined at any sampled point by the square root of the sum of the squares of the I and Q components:

$$M = \sqrt{{I}^{2} + {Q}^{2}} ; \qquad Eqn . 1$$

and the phase of the received magnetic resonance signal may also be determined according to the following relationship:

$$φ = {tan}^{- 1} ( \frac{Q}{I} ) . \qquad Eqn . 2$$

The pulse sequence server 110 also optionally receives patient data from a physiological acquisition controller 130. By way of example, the physiological acquisition controller 130 may receive signals from a number of different sensors connected to the patient, such as electrocardiograph (ECG) signals from electrodes, or respiratory signals from a respiratory bellows or other respiratory monitoring device. Such signals are typically used by the pulse sequence server 110 to synchronize, or “gate,” the performance of the scan with the subject's heartbeat or respiration.

The pulse sequence server 110 also connects to a scan room interface circuit 132 that receives signals from various sensors associated with the condition of the patient and the magnet system. It is also through the scan room interface circuit 132 that a patient positioning system 134 receives commands to move the patient to desired positions during the scan.

The digitized magnetic resonance signal samples produced by the RF system 120 are received by the data acquisition server 112. The data acquisition server 112 operates in response to instructions downloaded from the operator workstation 102 to receive the real-time magnetic resonance data and provide buffer storage, such that no data are lost by data overrun. In some scans, the data acquisition server 112 does little more than pass the acquired magnetic resonance data to the data processor server 114. However, in scans that require information derived from acquired magnetic resonance data to control the further performance of the scan, the data acquisition server 112 is programmed to produce such information and convey it to the pulse sequence server 110. For example, during prescans, magnetic resonance data are acquired and used to calibrate the pulse sequence performed by the pulse sequence server 110. As another example, navigator signals may be acquired and used to adjust the operating parameters of the RF system 120 or the gradient system 118, or to control the view order in which k-space is sampled.

The data processing server 114 receives magnetic resonance data from the data acquisition server 112 and processes it in accordance with instructions downloaded from the operator workstation 102. Such processing may, for example, include one or more of the following: reconstructing two-dimensional or three-dimensional images by performing a Fourier transformation of raw k-space data; performing other image reconstruction techniques, such as iterative or backprojection reconstruction techniques; applying filters to raw k-space data or to reconstructed images; generating functional magnetic resonance images; calculating motion or flow images; and so on.

Images reconstructed by the data processing server 114 are conveyed back to the operator workstation 102. Images may be output to operator display 112 or a display 136 that is located near the magnet assembly 124 for use by attending clinician. Batch mode images or selected real time images are stored in a host database on disc storage 138. When such images have been reconstructed and transferred to storage, the data processing server 114 notifies the data store server 116 on the operator workstation 102. The operator workstation 102 may be used by an operator to archive the images, produce films, or send the images via a network to other facilities.

The MRI system 100 may also include one or more networked workstations 142. By way of example, a networked workstation 142 may include a display 144, one or more input devices 146 (such as a keyboard and mouse or the like), and a processor 148. The networked workstation 142 may be located within the same facility as the operator workstation 102, or in a different facility, such as a different healthcare institution or clinic. The networked workstation 142 may include a mobile device, including phones or tablets.

The networked workstation 142, whether within the same facility or in a different facility as the operator workstation 102, may gain remote access to the data processing server 114 or data store server 116 via the communication system 140. Accordingly, multiple networked workstations 142 may have access to the data processing server 114 and the data store server 116. In this manner, magnetic resonance data, reconstructed images, or other data may be exchanged between the data processing server 114 or the data store server 116 and the networked workstations 142, such that the data or images may be processed remotely by a networked workstation 142. This data may be exchanged in any suitable format, such as in accordance with the transmission control protocol (TCP), the internet protocol (IP), or other known or suitable protocols.

As will be described, the MM system 100 can be used to acquire MR image data from the subject as the bladder is voided. To that end, a urine collection device 150 may be used that allows the subject to void the bladder into a reservoir 152.

In particular, as will be described, the systems and methods provided herein utilize three-dimensional (3D) MR data acquiring through a dynamic process involving the bladder, such as a bladder voiding process, to determine or calculate actual metrics that indicate the functionality of the bladder. For example, the metrics can include values for flow velocity and pressure at maximum flow velocity, as well as others. By calculating metrics directly from MR data (i.e., creating “calculated metrics”), as opposed to extrapolating metrics from interpolated data (i.e., creating “interpolated metrics”), the calculated metrics can be consistent with the current gold-standard for assessing bladder function, which is the multi-channel urodynamic study. Thus, the metrics can be readily correlated with traditional indexes for assessing urological function based on structural or functional information, such as outlet obstruction or contractility.

To investigate the potential to utilize MR to study the lower urinary tract, static volumetric (3D) MR images were acquired of the bladder at rest and upon voiding to then evaluate the changes that occurred. As one non-limiting example, a segmentation-based quantitative method was developed using Mimics 3-Matic, available from Materialise, Leuven, Belgium. In particular, in order to optimize the spatial resolution, a single-slab 3D FSE T2-weighted imaging sequence was used. This methodology utilized parallel imaging—simultaneous acceleration in two directions allowing the acquisition of additional and thinner slices, producing voxels with the same size in the slice and in-plane directions (isotropic resolution), contrary to conventional axial FSE acquisitions (anisotropic resolution, i.e., slice thickness larger than in-plane pixel size). 3D, static MRI was acquired both before and after voiding to allow for computational calculation of voiding dynamics metrics. These limited 3D MR images were used to create three-dimensional (3D) maps showing bladder contraction (wall displacement) estimated from computational interpolation between pre-and post-void MRI at four different time points during voiding.

Dynamic anatomical MRI has been used extensively for heart function assessment, where a balanced steady-state-free precession (bSSFP) pulse sequence has been used to create images that provide sufficient contrast to separate the myocardium and the blood pool. Data acquired using a bSSFP pulse sequence has previously been used to determine bladder motion during voiding in healthy volunteers. When attempting to use bSSFP pulse sequence for a bladder function study of a healthy volunteer during voiding, the volunteer expressed significant abdominal discomfort due to peripheral nerve stimulation during the real-time MR acquisition.

Instead, in accordance with the present disclosure a series of parallel, sagittal 2D dynamic real-time images can be acquired using a spoiled gradient echo (SGRE) pulse sequence to capture the bladder neck and urethra as well as other regions of the bladder. In this protocol, images can be acquired throughout for four minutes. Thus, images can be acquired of the full voiding mechanics during bladder voiding without inducing any discomfort or requiring interpolation.

These dynamic images can be segmented, for example, to measure the relative displacement of the bladder wall during voiding. While acquiring images throughout the voiding process, specific points in time may be isolated. For example, the pre and post voiding bladder volumes can be segmented and exported, for example, as stereolithography (STL) files. From 2D dynamic MRI data acquired during voiding, the area of a sagittal plane through the bladder can be measured over time. To estimate the motion of the bladder wall during voiding, for example, a spherical coordinate system can be defined for the bladder. The coordinate system origin can be set to be the center of the post voiding bladder volume. In general, the bladder wall displacement (d) is a three-dimensional vector that has spatial and time dependence given by, for example:

[dᵣ, d_{θ}, d_{ϕ}]=f(θ, ϕ, t) Eqn. 3.

The complete description of bladder wall motion can be simplified by assuming the bladder wall only moves radially (d_{θ}=d_{ϕ}=0) and the spatial and time dependence of the wall motion can be separated as:

dᵣ=d₀(θ, ϕ)α(t) Eqn. 4;

where d₀(θ,ϕ) is the total displacement from the pre- to post-void anatomies and α(t) is the time dependence function that varies from 0 at the start of voiding to 1 at the end of voiding. For wall displacement analysis, the bladder wall can be divided, for example, into anterior-posterior, dome-base, and left-right regions and an asymmetry ratio was calculated based on the difference between the median displacement of the left and right bladder wall regions. For each point on the bladder surface, d₀, the distance between the pre- and post-voiding anatomies can be calculated, for example, using a fast, minimum storage ray-triangle intersection algorithm. The time dependence function, α(t), can be calculated, for example, from real time measurements of bladder area during voiding, given by, for example:

$$∝ ( t ) = {( \frac{A ( t ) - A ( {t}_{0} )}{A ( {t}_{end} ) - A ( {t}_{0} )} )}^{\frac{1}{2}} ; \qquad Eqn . 5$$

where A(t) is the bladder area, t₀ is the time at the start of voiding, and t_{end} is the time at the end of voiding. Bladder area measurements from the real-time sagittal MR images showed a sigmoidal behavior. Based on that behavior, α(t) can be chosen to be a square root of cosine function, for example.

Using these mathematical and computational methods, a pattern and symmetry of bladder contraction can be determined and used to predict patters of urine flow and vortices within the bladder using modeling. For example, patient-specific bladder anatomies can be imported into the CFD software. One example of CFD software is CONVERGE v2.4 available from Convergent Science Inc, Madison, Wis. Bladder wall motion can be estimated as described above and imposed with a user-defined function to virtually drive voiding. The urethra wall can be assumed to be rigid and the urethra outlet was set to atmospheric pressure. Vorticity, the curl of the velocity field, can be averaged over the bladder volume (urethra not included). Dimensionless vorticity can then be calculated based on average urethra flow rates and the prostatic urethra diameter for each subject.

A variety of studies were performed. These studies complemented several other studies done using MRI imaging of the bladder before, during, and after voiding to describe changes in the bladder, bladder neck, and prostate during voiding. Like those previous studies, such studies and images significantly added to the understanding of bladder function during voiding but had no direct clinical value or import because they do not relate to clinical protocols.

In particular, these studies and the data underlying the studies did not yield metrics consistent with the current gold-standard for assessing bladder function, which is the multi-channel urodynamic study. That is, without metrics that can be readily correlated with traditional indexes for assessing urological function based on structural or functional information, such as outlet obstruction or contractility, the information is not applicable to current clinical workflows or care protocols.

On the other hand, the present disclosure provides systems and methods that utilize dynamic, volumetric 3D images, in specific imaging protocols, to produce, time-resolved 3D images of the voiding effort of the bladder, allowing the calculation of the pressure and flow during voiding without interpolation using CFD models. Measurements of bladder pressure and flow are normally made in clinical practice by multichannel urodynamics, using a small pressure-sensing catheter placed within the bladder and physical measurement of urine flow. The systems and methods provided herein are able to determine bladder pressure and flow measurements in a non-invasive fashion. A urine flow curve for the entire voiding effort can be created without relying on guessing or interpolating. Thus, maximum urine flow rate and the bladder pressure at maximum flow can be determined based on actual data and, thereby, consistent with the data-driven derivation of these metrics in multichannel urodynamics studies. That is, the present disclosure provides systems and methods to obtain the two metrics (normally obtained from multichannel urodynamics) that are central in the evaluation and treatment of men with lower urinary tract symptoms—the bladder contractility index (BCI) and bladder outlet obstruction index (BOOI).

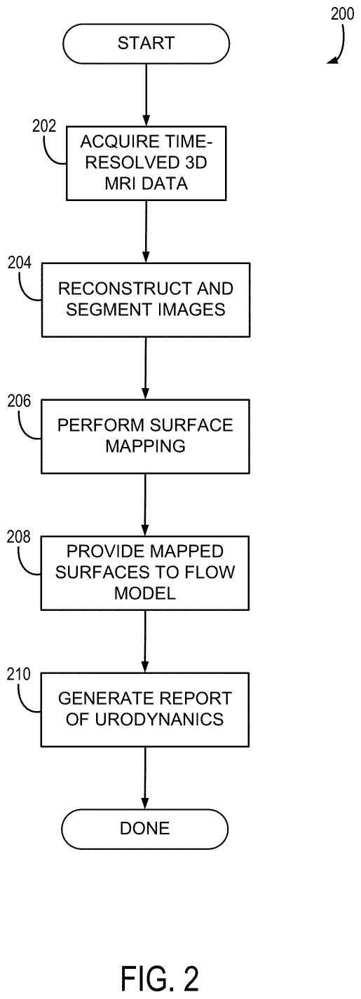

Specifically, referring to FIG. 2, a flow chart is provided that sets forth some example steps of a process 200 in accordance with the present disclosure. At process block 202, the process 200 begins with the acquisition of time-resolved, three-dimensional (3D), MR data from the bladder during voiding, such as using the MM system of FIG. 1. The time-resolved, 3D MR data is reconstructed into images that are segmented at process block 204. As such, a 3D object of the bladder at each MR time phase is produced. That is, from one MR scan session performed during a dynamic process, such as bladder voiding, a sequence of 3D objects showing the continuous change in bladder geometry during voiding are obtained produced. The objects can be processed or segmented such that the bladder wall is smooth, and the bladder neck is flat. In accordance with one example in accordance with the present disclosure, these 3D objects can be formed of discrete triangular elements that evolve over the entire dynamic process.

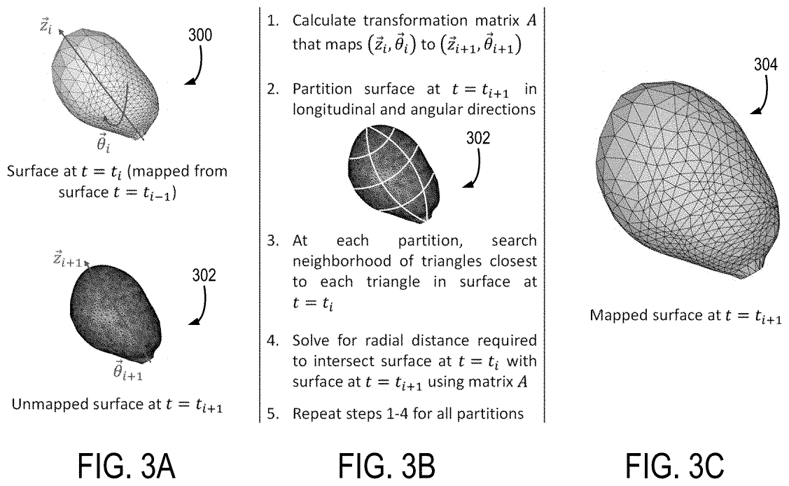

At process block 206, these segmented 3D objects extending over the dynamic process processed to map a surface of the bladder. In particular, referring to FIGS. 3A-3C, one non-limiting example of a process for producing surface files is illustrated. The surface files, together, can be formed into a sequence of surface files that represent the surface of the bladder wall at each time phase during the voiding event. These surfaces are formed by different number of triangular elements and of different topologies. To allow for processing across the time-series of images acquired from across the whole dynamic process (e.g., rather than just a point prior to voiding and a point after voiding), the surfaces are converted into surfaces that are consistent across the time-series of images. For example, in one, non-limiting implementation, consistency between images may be achieved by using the same number of triangular elements (or other shapes) and same topology between adjacent images or volumes, while preserving the shape of the bladder wall within a given image frame.

Referring to FIG. 3A, a given phase 300 of the bladder is selected for surface mapping to serve as a base surface that is the reference surface onto which all other surfaces are mapped onto. In one non-limiting example, the surface at the middle of the voiding sequence may be chosen to be the base surface, as opposed to a surface at the beginning or prior to voiding or at the end of voiding. The selected base surface is translated about a vector (\vec{z}ᵢ) such that the center of the bladder is at the origin. All the surfaces can be translated by the same vector to preserve the bladder's relative position during voiding.

Adjacent image volumes are processed. The selected surface at the given phase 300 is selected to act as a base surface (surface0), which defines the adjacent surface as surface1 from an adjacent phase 302. First, the coordinates of the vertices of the triangle elements of surface0 and surface1 are transformed from cartesian to cylindrical coordinate system. In one non-limiting example, the axial direction of the cylindrical coordinate system can be chosen as a vector from the bladder neck to the top of the bladder dome (\vec{z}ᵢ), such that translation is about qᵢ, as illustrated in FIG. 3A.

In particular, in the given phase 300, the axial vector for surface0 is \vec{z}₀ and, in the adjacent phase, the axial vector for surface1 is \vec{z}₁. In the adjacent phase 302, the surface can be petitioned in longitudinal and angular directions. A vector can be defined such that \vec{n}=\vec{z}₀×\vec{z}₁ and an angle can be defined such that angle α=arccos(\vec{z}₀·\vec{z}₁). Therefore, two cylindrical coordinate systems are defined, one for each surface. A transformation matrix, A, can be used to transform the coordinate system for surface0 to that of surface1, for example, where the matrix is given by:

$$A = [ \text{} \begin{matrix}\begin{matrix}cos ( α ) + \\ {n}_{1}^{2} ( 1 - cos ( α ) )\end{matrix} & \begin{matrix}{n}_{1} {n}_{2} ( 1 - cos ( α ) ) - \\ {n}_{3} sin ( α )\end{matrix} & \begin{matrix}{n}_{1} {n}_{3} ( 1 - cos ( α ) ) + \\ {n}_{2} sin ( α )\end{matrix} \\ \begin{matrix}{n}_{l} {n}_{2} ( 1 - cos ( α ) ) + \\ {n}_{3} sin ( α )\end{matrix} & \begin{matrix}cos ( α ) + \\ {n}_{2}^{2} ( 1 - cos ( α ) )\end{matrix} & \begin{matrix}{n}_{2} {n}_{3} ( 1 - cos ( α ) ) - \\ {n}_{1} sin ( α )\end{matrix} \\ \begin{matrix}{n}_{1} {n}_{3} ( 1 - cos ( α ) ) - \\ {n}_{2} sin ( α )\end{matrix} & \begin{matrix}{n}_{2} {n}_{3} ( 1 - cos ( α ) ) + \\ {n}_{1} sin ( α )\end{matrix} & \begin{matrix}cos ( α ) + \\ {n}_{3}^{2} ( 1 - cos ( α ) )\end{matrix}\end{matrix} ]$$

where

$$\vec{n} = [ \begin{matrix}{n}_{1} \\ {n}_{2} \\ {n}_{3}\end{matrix} ] .$$

With common coordinate systems, the surfaces can be binned. Referring to FIG. 3B, in this non-limiting example, surface1 can be divided into sections in the axial and azimuthal directions. In this example, surface1 of the adjacent phase 302 can be divided into, for example, 12-20 sections. In each section, the radial distance required to intersect each triangle element can be found in surface1 to the closest triangle in surface0. This can be achieved by solving a system of equations with the previously calculated transformation matrix, A. After solving for every triangle element, this process can be repeated for other sections. Binning reduces the computational demand by reducing the number triangles when searching for surface0's closest triangle. In this way, though optional, binning reduces the number of operations and runtime for the mapping algorithm.

These radial distances can be used to generate a new surface file. This is the mapped version of surface1, which can be referred to as surface1r 304, which is illustrated in FIG. 3C. Surface1r has the same number of triangle elements and topology as surface0. This process is then repeated between surface1r and surface2, where surface2 is the bladder wall surface from the successive MR time phase. These processes can be repeated for every surface or a select number of surfaces, to create mapped surfaces. Every mapped surface has the same number of triangle elements and topology, but the shapes are the same as the bladder wall at their corresponding time phase.

Referring again to FIG. 2, the process continues by providing the mapped surfaces to a flow model at process block 208. In one non-limiting example, the mapped surface are provided to a computational flow dynamics (CFD) model. In one particular, non-limiting example, the mapped surfaces can be loaded into a CFD model using CONVERGE, available from Convergent Science, Inc., Madison, Wis., USA, along with the timestamp for each surface. Regardless of the particular implementation, the CFD model may provide a wall motion driven CFD simulation, where the outlet is the bladder neck and the pressure can be controlled relative to the bladder neck. This general CFD model can be coupled with a CFD model of the urethra to impose the bladder neck pressure. In one further, non-limiting example, a pressure-implicit with splitting of operators (PISO) algorithm can be used as a Navier-Stokes equation solver. The dimensions of the cubic mesh elements can be controlled.

By using volumetric MR data acquired over the full dynamic process, the CFD models are designed provide comprehensive information on the dynamics of, for example, bladder voiding comparable to clinically available methods of multi-channel urodynamic studies that require direct and invasive measures of urodynamics. That is, a report is generated at process block 210 that is clinically consistent with those generated by invasive, multi-channel urodynamic studies.

For example, some non-limiting examples of metrics that are generated at process block 210 can include velocity, pressure, wall shear stress, and vorticity. Again, these metrics can be directly derived from the MR data over the whole voiding process, rather than extrapolated or estimated, for example, for a pre-void image and a post-void image. That is, regardless of the particular metrics, it is noted that the metrics produced are not interpolated or estimated from static or 2D images but can be derived directly from time-series volumetric images. As such, these metrics can be used to calculate useful indices used by urologists in clinical workflows, such as bladder outlet obstruction index (BOOT) and bladder contractility index (BCI). That is, because the metrics produced as described above are actually calculated directly from the MR data acquired over the dynamic process, metrics such as max flow and velocity at max flow can be determined with the accuracy and precision expected for use in clinical indices, such as BOOI and BCI, which have historically only been determined using max flow and velocity determined directly and invasively using multi-channel urodynamic studies.

Thus, a variety of metrics can be determined both at a given instance at over the entire voiding process, such as, for example, bladder capacity, voiding pressure, flow dynamics, pressure at maximum flow, post voiding residual volume, emptying efficiency, and maximum flow. Furthermore, these metrics can be readily used to calculate any of a variety of indices used in clinical decision making, such as BOOI and BCI.

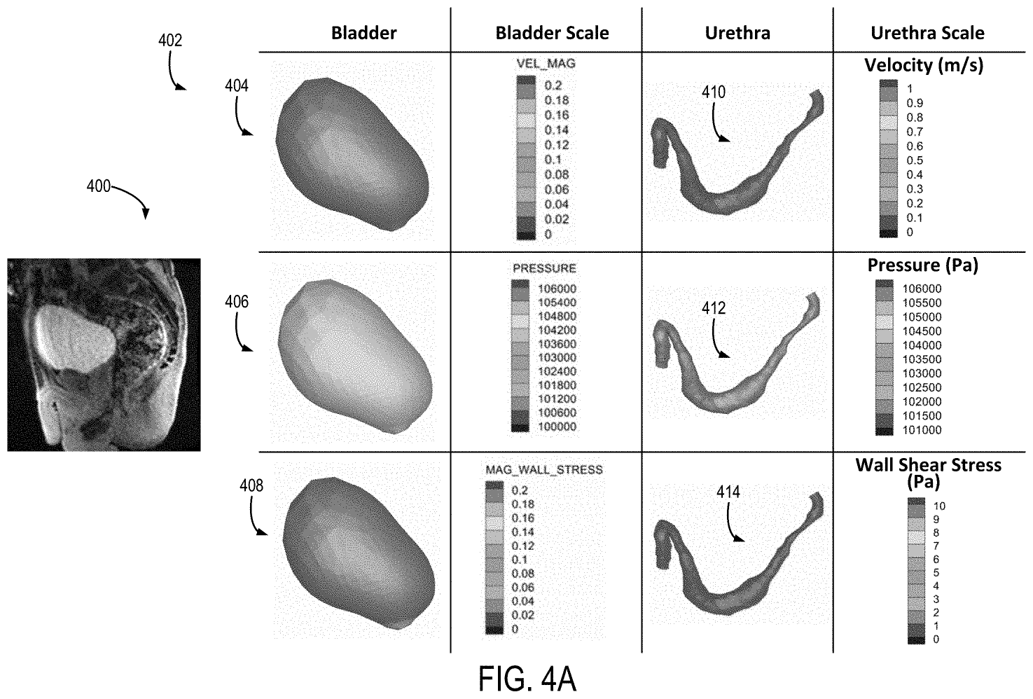

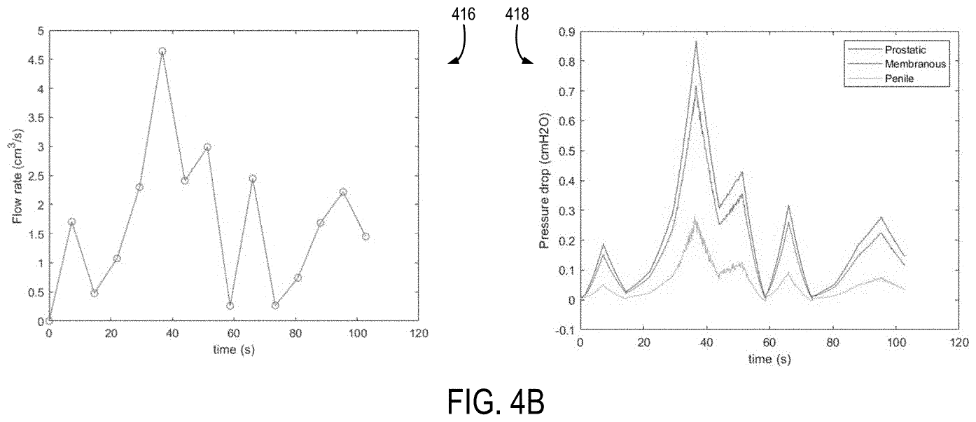

Additionally, the outputs of the CFD models may be used to create visualizations mapped to the volumetric movies created from the MR data. Such visualizations can also be provided as part of the reports generated at process block 210. For example, referring to FIG. 4A, a suite of information may be included in a report that includes images or movies of the anatomy 400, and/or images or movies 402 of the whole anatomy or portions of the anatomy with particular metrics illustrated in visualizations, such as coloration of 3D images or movies. In the non-limiting example illustrated in FIG. 4A, the bladder is isolated and illustrated over time with colorizations to show velocity 404, pressure 406, and wall shear stress 408. Additionally, in this non-limiting example, similar images or movies are provided for the urethra to show velocity 410, pressure 412, and stress 414.

Beyond movies and/or metrics registered to anatomical data, the metrics can be reported over time, such as illustrated in FIG. 4B. Again, because the above-described systems and methods facilitate the use of MR data acquired over the entire bladder voiding, any metric can be reported over time. For example, as illustrated in FIG. 4B, flow rate can be reported over time 416. As such, the actual maximum flow rate can be determined, not just estimated from interpolated data. Furthermore, the actual time of the maximum flow rate can be determined, not estimated. Such metrics can be reported for the entire system or separated into particular anatomy, such as bladder versus urethra, which is not possible with interpolated data or even using multichannel studies. For example, the pressure drop can be shown over time 418 for penile, membranous, and prostatic data.

Irrespective of the particular information or way of presenting information within a given report, the systems and methods provided herein are able to provide the clinical information currently only available to clinicians via multichannel urodynamic studies. However, the systems and methods described herein are non-invasive and, for the first time, provides detailed anatomic and functional information on the lower urinary tract during the entire voiding cycle. A variety of additional information not previously available to clinicians facilitates a deeper understanding of impaired contractility by identifying and characterizing specific putative causes such as global hypokinesis, dyscoordination of bladder contraction, or loss of bladder power due to intravesical flow vortices.

Thus, the systems and methods provided herein comprehensively characterize the bladder and lower urinary tract biomechanics by using MRI based computational fluid dynamics. No changes to the scanner hardware or acquisition algorithms are required. The MR data may be processed locally or sent to a remote location to perform segmentation, analysis, and reporting. In any case, a report is generated for the clinician that provides direct measures typically only available via multichannel urodynamic studies, but also provides substantial information that can better inform clinical decisions.

Additionally, while the above described systems and methods focused on “voiding,” the systems and methods also readily apply to bladder filling. That is, some studies suggest that bladder filling is an active process (not just passive filling but actual bladder muscle accommodation and possible micro-motions). In addition, many patients with LUTS experience so-called “involuntary bladder contractions that produce symptoms or urinary urgency and/or urge incontinence. Similarly, some women experience so-called stress incontinence that is precipitated by a Valsalva maneuver, such as coughing or straining. These and other dynamics can readily be studied, imaged, and/or included in reports using the above described systems and methods.

The invention has been described according to one or more preferred embodiments, and it should be appreciated that many equivalents, alternatives, variations, and modifications, aside from those expressly stated, are possible and within the scope of the invention.

The preceding discussion is presented to enable a person skilled in the art to make and use embodiments of the invention. Various modifications to the illustrated embodiments will be readily apparent to those skilled in the art, and the generic principles herein can be applied to other embodiments and applications without departing from embodiments of the invention. Thus, embodiments of the invention are not intended to be limited to embodiments shown, but are to be accorded the widest scope consistent with the principles and features disclosed herein. The detailed description is to be read with reference to the figures, in which like elements in different figures have like reference numerals. The figures, which are not necessarily to scale, depict selected embodiments and are not intended to limit the scope of embodiments of the invention. Skilled artisans will recognize the examples provided herein have many useful alternatives and fall within the scope of embodiments of the invention.

It is to be understood that the disclosure is not limited in its application to the details of construction and the arrangement of components set forth in the description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

We claim:

1. A magnetic resonance (MR) imaging system comprising:
a magnet system configured to generate a static magnetic field (B₀) about at least a portion of a subject including a urinary tract;
a plurality of gradient coils configured to apply magnetic gradients to the static magnetic field;
a radio frequency (RF) system configured to apply an excitation field to the subject and acquire time-resolved, three-dimensional (3D) MR image data from the urinary tract of the subject;
a computer system programmed to:
control the plurality of gradient coils and the RF system to acquire the time-resolved 3D MR image data from the urinary tract as a bladder of the urinary tract begins, continues through, and completes a dynamic process including a bladder;
reconstruct the time-resolved 3D MR image data to produce volumetric, time-resolved images of the urinary tract;
segment the volumetric, time-resolved images of the urinary tract to identify anatomical structures of the urinary tract;
perform a surface mapping of the anatomical structures to produce a consistent set of mapped anatomical structures across the volumetric, time-resolved images;
process the consistent set of mapped anatomical structures using a flow model to calculate metrics describing a function of the urinary tract during the dynamic process;
generate a report using the metrics describing function of the urinary tract during the dynamic process; and
display the report for clinical analysis of the function of the urinary tract.

2. The system of claim 1, wherein the computer system is further configured to define a series of structural element shapes across a segmented bladder in a given image frame in the volumetric, time resolved images and utilize a common number of structural element shapes across each image frame in the volumetric, time resolved images.

3. The system of claim 2, wherein the computer system is configured to utilize triangles as the structural element shape and enforce a consistent number of triangles across each image frame in the volumetric, time resolved images, while preserving a shape of the bladder wall within each image frame.

4. The system of claim 1, wherein the computer system is further configured to utilize a common coordinate system between image frames in the volumetric, time resolved images to bin segmented surfaces in the volumetric, time-resolved images to increase computational efficiency of the surface mapping.

5. The system of claim 1, wherein the computer system is further configured to determine at least one of bladder capacity, voiding pressure, flow dynamics, pressure at maximum flow, post voiding residual volume, emptying efficiency, or maximum flow to determine the metrics describing function of the urinary tract during the dynamic process.

6. The system of claim 1, wherein, to generate the report, the computer system is configured to produce at least one of images with functional overlays, graphs showing metrics over time, or metric-correlated indices including at least one of bladder outlet obstruction index (BOOT) or bladder contractility index (BCI).

7. A method comprising:
receiving time-resolved images of a urinary tract of a subject as a bladder of the urinary tract begins, continues through, and completes a dynamic process including a bladder;
segmenting the time-resolved images of the urinary tract to identify boundaries of anatomical structures of the urinary tract;
performing a surface mapping of the boundaries of the anatomical structures to produce a consistent set of mapped anatomical structures across the time-resolved images;
using a flow model and the consistent set of mapped anatomical structures, calculating metrics describing function of the urinary tract during the dynamic process; and
generating a report using the metrics describing function of the urinary tract during the dynamic process.

8. The method of claim 7, further comprising defining a series of structural elements across a segmented bladder in a given image frame in the time resolved images and utilize a common number of structural elements across each image frame in the time-resolved images.

9. The method of claim 8, wherein the structural elements are triangles and further comprising enforcing a consistent number of triangles across each image frame in the time resolved images, while preserving a shape of the bladder wall within each image frame.

10. The method of claim 7, further comprising utilizing a common coordinate system between image frames in the time resolved images to bin segmented surfaces in the time-resolved images to increase computational efficiency of the surface mapping.

11. The method of claim 7, further comprising determining at least one of bladder capacity, voiding pressure, flow dynamics, pressure at maximum flow, post voiding residual volume, emptying efficiency, or maximum flow to determine the metrics describing function of the urinary tract during the dynamic process.

12. The method of claim 7, further comprising producing at least one of images with functional overlays, graphs showing metrics over time, or metric-correlated indices including at least one of bladder outlet obstruction index (BOOI) or bladder contractility index (BCI).

13. The method of claim 7, wherein the time-resolved images are volumetric magnetic resonance images.
